(19) United States
(12) Patent Application Publication
(10) Pub. No.: US 2011/0206778 A1
(43) Pub. Date: Aug. 25, 2011

(54) Treatment For Neuropathy, Shingles And Related Disorders

(76) Inventor: Ronald Bourgeois, Hillsboro Beach, FL (US)

(21) Appl. No.: 12/712,551

(22) Filed: Feb. 25, 2010

(51) Int. Cl. A61K 33/30; A61P 17/00; A61P 25/02; A61P 31/22

(52) U.S. Cl. 424/642

(57) ABSTRACT

A zinc oxide treatment for neuropathy, shingles, dermatitis and shingles. The treatment is applied as a cream having an active ingredient of approximately 25%, or more, zinc oxide mixed with a suitable excipient such as baby oil or petroleum jelly. The zinc oxide acts as a therapeutic agent, a sun blocking agent, and a topical anesthetic that allows the effected area to be protected.

### BACKGROUND

1. Technical Field

This invention relates in general to creams, ointments, salves and solutions a topical treatment of inflamed areas of skin, and related disorders. In particular, this invention relates to treatment of neuropathy, shingles, dermatitis, and a variety of the Herpes virus, including Herpes Zoster, Herpes Simplex I, and Herpes Simplex 2. The topical ointment comprises an active ingredient of approximately 25% or more zinc oxide mixed with a suitable excipient or such as baby oil or petroleum jelly.

2. Background of the Invention

As many people have unfortunately become aware, neuropathy is a medical condition that can significantly interfere with an individual's day-to-day life. Neuropathy can take several forms. One form (peripheral neuropathy) damages nerves of the peripheral nervous system, and can be caused by disease or other illness.

Numerous dermatological preparations are known in the prior art. For example the Physician's Desk Reference (PDR) lists numerous categories of dermatological preparations, typically for antibacterial and antifungal preparations. Anti-inflammatory agents typically utilize a corticosteroid as their active ingredient. However, these preparations do not heal the inflammations produced by the Herpes viruses. Merck's Manual of Diagnosis and Therapy suggests for Herpes Simplex topical use of Idoxuridine (IDU) for herpetic keratitis (eye infection), but this material is not found in PDR. Merck mentions Acyclovir as having shown promise in the treatment of Herpes. For other Herpes lesions, Merck suggests “drying lotions” such as Camphor Spirit or 70% alcohol, and for Herpes Zoster, Merck states “there is no known specific therapy.” Hormones represent another class of anti-inflammatory compounds. However, no known hormones have produced a satisfactory response in the treatment of Herpes inflammation or lesions.

In many topical ointments, zinc oxide is frequently used. However, it is used as a secondary component, and not the active agent that produces the healing effect. In the prior art, zinc oxide is used as an excipient, filler, surfactant, or sun-blocking agent. To date, the prior art has not produced a topical treatment in which zinc oxide, having sufficiently high concentration levels, has not been used as the active ingredient the treatment of these disorders. Rather, it is typically used as a secondary component that is there to facilitate effectiveness of the primary active ingredients by acting as an excipient, a sun block, or bulk filler.

While the prior art has provided a variety of topical treatments for many ailments, it has failed to provide a safe topical ointment that is based on the use of high concentrations of zinc oxide as the active ingredient for the treatment of neuropathy, shingles, or other herpes related ailments. The ointment provided by the invention uses high concentration levels of zinc oxide, on the order of approximately twenty five percent (25%) or more, as the sole active ingredient in the treatment of these disorders. The zinc oxide is used in conjunction with excipients, such as baby oil or petroleum jelly to facilitate spreading and application of the zinc oxide onto the surface of the patient's skin.

### SUMMARY OF THE INVENTION

This invention provides a zinc oxide treatment for shingles and neuropathy. The treatment is applied as a cream having an active ingredient of approximately 25%, or more, zinc oxide mixed with a suitable excipient such as baby oil or petroleum jelly. The zinc oxide acts as simultaneously as a therapeutic agent, a sun-blocking agent, and a topical anesthetic.

### DETAILED DESCRIPTION OF THE PREFERRED EMBODIMENT

The invention is a topical ointment for application to skin surface areas suffering from neuropathy, shingles, or other viral infections such as various Herpes types. In the preferred embodiment, zinc oxide is used as the active ingredient in the treatment of these ailments. The topical ointment provided by the invention uses high concentration doses of zinc oxide on the order of approximately twenty five, or more, percent (25%) or more. The remaining component of the ointment is an excipient to allow the zinc oxide to be spread easily on the patient's skin, and to help secure the zinc oxide to the patient's skin. The excipient can be any suitable material, such as commercially available baby oil, a petroleum jelly such as Ethylene™, etc.

The zinc oxide ointment provides several functions. Most important, it acts as a therapeutic agent for the treatment of shingles, herpes, etc. Secondly, zinc oxide acts as a sunscreen to protect skin. Thirdly, it provides a topical anesthetic effect to soothe an infected area. The ointment does not heal shingles or herpes. What it does is provide an effective method of reducing pain and discomfort associated with these conditions while the body heals itself. It is been found that the zinc oxide is more effective when applied in high concentration levels of approximately 25% or more. Of course, a portion of the ointment requires the use of an excipient to allow the zinc oxide to be easily applied to skin surfaces.

The ointment can be used for a variety of conditions. For example, individuals with restless leg syndrome often have difficulty sleeping due to a pins-and-needles sensation in their legs. The ointment is effective in reducing this sensation so that individuals can sleep better.

Application of the zinc oxide ointment should be applied to the entire effected area with one exception: it should not be applied directly to open wounds. Further, the effected area should be periodically cleaned and then provided with a new application of the zinc oxide ointment. This process should be repeated as needed. Typically, the preferred application time would be from one to seven days, but can vary based on individual health, metabolism, etc.

A preferred method of applying the ointment is to apply the ointment to an effected area and keep it on as long as possible. For example, if an individual's foot is being treated, the ointment would be applied, and then the foot would be covered with a sock. The purpose of the sock would be to keep the ointment on and in contact with the skin as long as possible. Further, since the ointment is inexpensive, it allows the patient to shower when convenient and then reapply a fresh layer of ointment.

An advantage of the invention is that it can be inexpensively provided to the public as a nonprescription treatment that can be applied directly by the patient. In an era of high health care costs, the ability to provide an inexpensive method of treatment carries significant advantages for the ordinary consumer.

Those skilled in the art will recognize that various changes can be made any ingredients used in the zinc oxide ointment. Any suitable excipient can be used in combination with zinc oxide. Likewise, the percentage of zinc oxide can be varied to any suitable amount so long as its ability to be conveniently spread on skin surface, and to remain adhered to skin surface, is not impacted. Further, the viscosity of a topical ointment can be varied. For example, it can be a thick paste, or an easily spreadable cream. The only requirement is that it thoroughly covers the effected area and does not inadvertently separate from the skin surface.

Another advantage of the invention is that it does not require any drugs or other ingredients. The zinc oxide only requires a suitable excipient to allow it to be applied. The excipient can be baby oil, petroleum jelly, or any other suitable excipient cream or a combination of any of the foregoing.

In an alternative embodiment, the ointment can be made from an excipient, such as baby oil or petroleum jelly, zinc oxide as the active ingredient for pain relief, and capsaicin and/or aspercreme for pain.

While specific embodiments have been discussed to illustrate the invention, it will be understood by those skilled in the art that variations in the embodiments can be made without departing from the spirit of the invention. For example, the proportion of ingredients can vary, the type of excipients can vary, bulking agents can be ended, etc. Therefore, the invention shall be limited solely to the scope of the claims.

I claim:

1. A topical ointment for the treatment of skin disorders, comprising:
an active ingredient of zinc oxide comprising approximately 25% or more of the ointment; and
an excipient for facilitating application of the zinc oxide to in effect an area of skin.

2. A topical ointment, as in claim 1, wherein:
the excipient is baby oil.

3. A topical ointment, as in claim 2, wherein:
the ratio of zinc oxide to excipient, when mixed, results in an ointment having the consistency of a paste.

4. A topical ointment, as in claim 2, wherein:
the ratio of zinc oxide to excipient, when mixed, results in an ointment having the consistency of a cream.

5. A topical ointment, as in claim 1, wherein:
the excipient is petroleum jelly.

6. A topical ointment, as in claim 5, wherein:
the ratio of zinc oxide to excipient, when mixed, results in an ointment having the consistency of a paste.

7. A topical ointment, as in claim 5, wherein:
the ratio of zinc oxide to excipient, when mixed, results in an ointment having the consistency of a cream.

8. A method of treating neuropathy, shingles, and herpes, including the steps of:
spreading a topical ointment having an active ingredient of approximately 25% or more zinc oxide, and an excipient on an effected area of a patient's skin.

9. A method, as in claim 8, including the additional step of:
using baby oil is the excipient.

10. A method, as in claim 9, including the additional steps of:
periodically removing the topical ointment from the effected area;
cleaning the effected area;
reapplying the topical ointment.

11. A method, as in claim 10, wherein:
the topical ointment uses a ratio of zinc oxide to excipient that provides an ointment having the consistency of a paste.

12. A method, as in claim 10, wherein:
the topical ointment uses a ratio of zinc oxide to excipient that provides an ointment having the consistency of a cream.

13. A method, as in claim 8, including the additional step of:
using petroleum jelly as the excipient.

14. A method, as in claim 13, wherein:
the topical ointment uses a ratio of zinc oxide to excipient that provides an ointment having the consistency of a paste.

15. A method, as in claim 14, including the additional step of:
periodically removing the topical ointment from the effected area;
cleaning the effected area;
reapplying the topical ointment.

16. A method, as in claim 13, wherein:
the topical ointment uses a ratio of zinc oxide to excipient that provides an ointment having the consistency of a cream.

17. A method, as in claim 16, including the additional step of:
periodically removing the topical ointment from the effected area;
cleaning the effected area;
reapplying the topical ointment.
